SEQUENCE LISTING (24 sequences)

SEQ ID NO: 1 - DNA, 1557 bp, Escherichia coli
  gene (1)..(1557): gshA
ttgatcccggacgtatcacaggcgctggcctggctggaaaaacatcctcaggcgttaaaggggatacagcgtgggctggagcgcgaaactttgcgtgttaatgctgatggcacactggcaacaacaggtcatcctgaagcattaggttccgcactgacgcacaaatggattactaccgattttgcggaagcattgctggaattcattacaccagtggatggtgatattgaacatatgctgacctttatgcgcgatctgcatcgttatacggcgcgcaatatgggcgatgagcggatgtggccgttaagtatgccatgctacatcgcagaaggtcaggacatcgaactggcacagtacggcacttctaacaccggacgctttaaaacgctgtatcgtgaagggctgaaaaatcgctacggcgcgctgatgcaaaccatttccggcgtgcactacaatttctctttgccaatggcattctggcaagcgaagtgcggtgatatctcgggcgctgatgccaaagagaaaatttctgcgggctatttccgcgttatccgcaattactatcgtttcggttgggtcattccttatctgtttggtgcatctccggcgatttgttcttctttcctgcaaggaaaaccaacgtcgctgccgtttgagaaaaccgagtgcggtatgtattacctgccgtatgcgacctctcttcgtttgagcgatctcggctataccaataaatcgcaaagcaatcttggtattaccttcaacgatctttacgagtacgtagcgggccttaaacaggcaatcaaaacgccatcggaagagtacgcgaagattggtattgagaaagacggtaagaggctgcaaatcaacagcaacgtgttgcagattgaaaacgaactgtacgcgccgattcgtccaaaacgcgttacccgcagcggcgagtcgccttctgatgcgctgttacgtggcggcattgaatatattgaagtgcgttcgctggacatcaacccgttctcgccgattggtgtagatgaacagcaggtgcgattcctcgacctgtttatggtctggtgtgcgctggctgatgcaccggaaatgagcagtagcgaacttgcctgtacacgcgttaactggaaccgggtgatcctcgaaggtcgcaaaccgggtctgacgctgggtatcggctgcgaaaccgcacagttcccgttaccgcaggtgggtaaagatctgttccgcgatctgaaacgcgtcgcgcaaacgctggatagtattaacggcggcgaagcgtatcagaaagtgtgtgatgaactggttgcctgcttcgataatcccgatctgactttctctgcccgtatcttaaggtctatgattgatactggtattggcggaacaggcaaagcatttgcagaagcctaccgtaatctgctgcgtgaagagccgctggaaattctgcgcgaagaggattttgtagccgagcgcgaggcgtctgaacgccgtcagcaggaaatggaagccgctgataccgaaccgtttgcggtgtggctggaaaaacacgcctga

SEQ ID NO: 2 - protein, 518 aa, Escherichia coli
  PEPTIDE (1)..(518): GshA
MIPDVSQALAWLEKHPQALKGIQRGLERETLRVNADGTLATTGHPEALGSALTHKWITTDFAEALLEFITPVDGDIEHMLTFMRDLHRYTARNMGDERMWPLSMPCYIAEGQDIELAQYGTSNTGRFKTLYREGLKNRYGALMQTISGVHYNFSLPMAFWQAKCGDISGADAKEKISAGYFRVIRNYYRFGWVIPYLFGASPAICSSFLQGKPTSLPFEKTECGMYYLPYATSLRLSDLGYTNKSQSNLGITFNDLYEYVAGLKQAIKTPSEEYAKIGIEKDGKRLQINSNVLQIENELYAPIRPKRVTRSGESPSDALLRGGIEYIEVRSLDINPFSPIGVDEQQVRFLDLFMVWCALADAPEMSSSELACTRVNWNRVILEGRKPGLTLGIGCETAQFPLPQVGKDLFRDLKRVAQTLDSINGGEAYQKVCDELVACFDNPDLTFSARILRSMIDTGIGGTGKAFAEAYRNLLREEPLEILREEDFVAEREASERRQQEMEAADTEPFAVWLEKHA

SEQ ID NO: 3 - DNA, 951 bp, Escherichia coli
  gene (1)..(951): gshB
atgatcaagctcggcatcgtgatggaccccatcgcaaacatcaacatcaagaaagattccagttttgctatgttgctggaagcacagcgtcgtggttacgaacttcactatatggagatgggcgatctgtatctgatcaatggtgaagcccgcgcccatacccgcacgctgaacgtgaagcagaactacgaagagtggttttcgttcgtcggtgaacaggatctgccgctggccgatctcgatgtgatcctgatgcgtaaagacccgccgtttgataccgagtttatctacgcgacctatattctggaacgtgccgaagagaaagggacgctgatcgttaacaagccgcagagcctgcgcgactgtaacgagaaactgtttaccgcctggttctctgacttaacgccagaaacgctggttacgcgcaataaagcgcagctaaaagcgttctgggagaaacacagcgacatcattcttaagccgctggacggtatgggcggcgcgtcgattttccgcgtgaaagaaggcgatccaaacctcggcgtgattgccgaaaccctgactgagcatggcactcgctactgcatggcgcaaaattacctgccagccattaaagatggcgacaaacgcgtgctggtggtggatggcgagccggtaccgtactgcctggcgcgtattccgcaggggggcgaaacccgtggcaatctggctgccggtggtcgcggtgaacctcgtccgctgacggaaagtgactggaaaatcgcccgtcagatcgggccgacgctgaaagaaaaagggctgatttttgttggtctggatatcatcggcgaccgtctgactgaaattaacgtcaccagcccaacctgtattcgtgagattgaagcagagtttccggtgtcgatcaccggaatgttaatggatgccatcgaagcacgtttacagcagcagtaa

SEQ ID NO: 4 - protein, 316 aa, Escherichia coli [sequence omitted]

SEQ ID NO: 5 - DNA, 70 bp, Artificial Sequence
  Oligonukleotid gshB-del-for
atgatcaagctcggcatcgtgatggaccccatcgcaaacatcaacatcaagtgtaggctggagctgcttc

SEQ ID NO: 6 - DNA, 70 bp, Artificial Sequence
  Oligonukleotid gshB-del-rev
ttactgctgctgtaaacgtgcttcgatggcatccattaacattccggtgaccgtcgacctgcagttcgaa

SEQ ID NO: 7 - DNA, 30 bp, Artificial Sequence
  Oligonukleotid gshAp-gshA-for
agtctctagaacgttatccttgggcgtttc

SEQ ID NO: 8 - DNA, 32 bp, Artificial Sequence
  Oligonukleotid gshAp-gshA-rev
agcttctagattgtaggcctgcacatatggtc

SEQ ID NO: 9 - DNA, 29 bp, Artificial Sequence
  Oligonukleotid gshA-OE-for
cccgaattcatgatcccggacgtatcaca

SEQ ID NO: 10 - DNA, 29 bp, Artificial Sequence
  Oligonukleotid gshA-OE-rev
aaaagatcttcaggcgtgtttttccagcc

SEQ ID NO: 11 - DNA, 32 bp, Artificial Sequence
  Oligonukleotid gshA-EcoRImut-for
tcaccatccactggtgtaataaattccagcaa

SEQ ID NO: 12 - DNA, 30 bp, Artificial Sequence
  Oligonukleotid gshA-EcoRImut-rev
attacaccagtggatggtgatattgaacat

SEQ ID NO: 13 - DNA, 32 bp, Artificial Sequence
  Oligonukleotid gshA-BglIImut-for
cgtttcagatcgcggaacaggtctttacccac

SEQ ID NO: 14 - DNA, 30 bp, Artificial Sequence
  Oligonukleotid gshA-BglIImut-rev
ctgttccgcgatctgaaacgcgtcgcgcaa

SEQ ID NO: 15 - DNA, 31 bp, Artificial Sequence
  Oligonukleotid tufBp-for
agtctctagacaatgatgttgaaaaagtgtg

SEQ ID NO: 16 - DNA, 49 bp, Artificial Sequence
  Oliginukleotid tufBp-rev
agcttctagaagatcttacggcgaattccaagtagcgcgcattctatgg

SEQ ID NO: 17 - DNA, 36 bp, Artificial Sequence
  Oligonukleotid tufBp-RBSopt-for
gaagatcttacggcgaattcctcctgcgcgcattct

SEQ ID NO: 18 - DNA, 30 bp, Artificial Sequence
  Oligonukleotid tufBp-RBSopt-rev
gaattcgccgtaagatcttctagatttcag

SEQ ID NO: 19 - DNA, 32 bp, Artificial Sequence
  Oligonukleotid tufBp-10opt-for
gaattcctcctgcgcgcattatatggagacat

SEQ ID NO: 20 - DNA, 30 bp, Artificial Sequence
  Oligonukleotid tufBp-10opt-rev
aatgcgcgcaggaggaattcgccgtaagat

SEQ ID NO: 21 - DNA, 34 bp, Artificial Sequence
  Oligonukleotid tufBp-35opt-for
atatggagacatgcgagttctgtcaactaaaaaa

SEQ ID NO: 22 - DNA, 30 bp, Artificial Sequence
  Oligonukleotid tufBp-35opt-rev
gaactcgcatgtctccatataatgcgcgca

SEQ ID NO: 23 - DNA, 27 bp, Artificial Sequence
  Oligonukleotid serA-NcoI-for
agtcccatggcgcagatgaaatcaacg

SEQ ID NO: 24 - DNA, 30 bp, Artificial Sequence
  Oligonukleotid serA-SacI-rev
tcaggagctcttagtacagcagacgggcgc

(19) United States
(12) Patent Application Publication
(10) Pub. No.: US 2014/0342399 A1
(43) Pub. Date: Nov. 20, 2014

(54) MICROORGANISM AND METHOD FOR OVERPRODUCTION OF GAMMA-GLUTAMYLCYSTEINE AND DERIVATIVES OF THIS DIPEPTIDE BY FERMENTATION

(71) Applicant: Wacker Chemie AG, Muenchen (DE)

(72) Inventors: Marcel THOEN, Muenchen (DE); Thomas SCHLOESSER, Burghausen (DE)

(73) Assignee: Wacker Chemie AG, Muenchen (DE)

(21) Appl. No.: 14/253,212

(22) Filed: Apr. 15, 2014

(30) Foreign Application Priority Data
May 17, 2013 (DE) 10 2013 209 274.1

(51) Int. Cl. C07K 5/062

(52) U.S. Cl. CPC C07K 5/06034
USPC 435/69.1; 435/252.33; 435/252.3

(57) ABSTRACT

The invention relates to a prokaryotic microorganism strain capable of overproducing γ-glutamylcysteine and its derivatives bis-γ-glutamylcystine and γ-glutamylcystine, which can be prepared from a parent strain, wherein said strain has a reduced cellular glutathione synthetase activity compared to the parent strain, and has a cellular γ-glutamylcysteine synthetase activity which is more greatly increased than in a strain with similarly reduced cellular glutathione synthetase activity.

### SEQUENCE LISTING STATEMENT

The Sequence Listing is filed in this application in electronic format only and is incorporated by reference herein. The sequence listing text file “W115420100_SeqLst_ST25” was created on Mar. 24, 2014, and is 15.1 KB in size.

### BACKGROUND OF THE INVENTION

The invention relates to a prokaryotic microorganism strain, which is suitable for overproduction of γ-glutamylcysteine (γGC) and the derivatives of this dipeptide, γ-glutamylcystine and bis-γ-glutamylcystine, and also methods for preparing these compounds.

γ-Glutamylcysteine is a dipeptide, which is formed in cells of prokaryotic and eukaryotic organisms by linking the amino acids L-cysteine and L-glutamic acid.

Bis-γ-glutamylcystine is a disulfide, which is formed by the oxidation of two molecules of γ-glutamylcysteine. This reaction is reversible, which means that bis-γ-glutamylcystine may be converted back to γ-glutamylcysteine by reduction (e.g. enzymatically or chemically).

γ-Glutamylcystine is a disulfide, which is formed by the oxidation of one molecule of γ-glutamylcysteine with one molecule of L-cysteine. This reaction is reversible, which means that γ-glutamylcystine may be converted back to γ-glutamylcysteine and L-cysteine by reduction (e.g., chemically).

The thiol compound γ-glutamylcysteine serves as the precursor of glutathione in numerous organisms. The first step of glutathione biosynthesis is the linking of a γ-peptide bond between the α-amino group of L-cysteine and the γ-carboxy group of L-glutamic acid by the ATP-dependent enzyme γ-glutamylcysteine synthetase. This reaction, moreover, is the limiting step in glutathione biosynthesis, since the enzyme γ-glutamylcysteine synthetase is inhibited by glutathione. The second step in glutathione biosynthesis is the reaction of L-glycine with γ-glutamylcysteine to give glutathione, in which L-glycine is linked via an α-peptide bond to the cysteinyl function of γ-glutamylcysteine. This likewise ATP-dependent reaction is catalyzed by the enzyme glutathione synthetase.

Due to its antioxidant properties, glutathione plays a key role in cells and participates in numerous cellular processes as a reducing agent, cosubstrate and cofactor. Glutathione-deficient organisms are, however, also known, in which the glutathione precursor, γ-glutamylcysteine, takes on the biological role of glutathione. Known examples are representatives of the halophilic archaebacteria such as Halobacterium halobium (Sundquist and Fahey, 1989, J. Biol. Chem. 264: 719-725).

In addition, different glutathione-deficient microorganism strains of various species are described in the literature, e.g. Saccharomyces cerevisiae (Grant et al., 1997, Mol. Biol. Cell. 8: 1699-1707), Synechocystis sp. (Cameron and Pakrasi, 2011, Plant Signal. Behay. 6: 89-92) or Eschericha coli (Fuchs and Warner, 1975, J. Bacteriol. 124: 140-148). These strains have mutations or deletions in the corresponding gene encoding glutathione synthetase (e.g. gshB for E. coli and Synechocystis sp. or gsh2 for S. cerevisiae). By modification or loss of the glutathione synthetase activity, these strains are no longer able, or are only able to a limited degree, to synthesize glutathione. Consequently, the cellular γ-glutamylcysteine level increases. These mutant strains are viable, since the enhanced formation and elevated concentration of γ-glutamylcysteine now present takes over numerous glutathione-specific functions. However, they are often characterized by an increased susceptibility towards reactive oxygen species and heavy metal ions in comparison to the corresponding wild-type strains such that their growth is partially impaired (Cameron and Pakrasi, 2011 Plant Signal. Behay. 6: 89-92; Helbig et al., 2008, J. Bacteriol. 190: 5439-5454).

The detoxification of reactive oxygen species, heavy metal ions and xenobiotics very frequently occurs in many organisms, also in man, inter alia, by means of glutathione (Grant et al., Mol. Biol. Cell. 8: 1699-1707). A low glutathione level is, moreover, often a symptom and/or the basis of various diseases such as autism, Parkinson's, cystic fibrosis, HIV, cancer or schizophrenia (Wu et al., 2004, J. Nutr. 134: 489-492). Therefore, there is considerable interest in maintaining a constant intracellular glutathione level or even to increase it. γ-Glutamylcysteine has already been successfully tested as a promising substance for increasing the cellular glutathione level (Anderson and Meister, 1983, P.N.A.S. 80: 707-711).

WO2006102722 describes an enzymatic method for preparing γ-glutamylcysteine, which is based on the reaction of cysteine derivatives and a γ-glutamyl donor by means of an immobilized γ-glutamylcysteine transpeptidase.

Microbial γ-glutamylcysteine producers are mainly fungal strains of the order Saccharomycetales, such as Saccharomyces cerevisiae or Candida utilis, but which are primarily used for the production of glutathione. Such strains are disclosed, for example, in W02010116833A1, U.S. Pat. No. 7,371,557B2 and US2005074835A1. Other fungal strains of the same order having an exceptionally high capacity for γ-glutamylcysteine production are described, inter alia, in EP2251413A1, U.S. Pat. No. 7,569,360B2, US2004214308A1, US2003124684A1, U.S. Pat. No. 7,410,790B2 and EP1489173B1. These fungal strains have a reduced glutathione synthetase activity as a common feature, besides various strain-specific characteristics, such as cerulenin or nitrosoguanidine resistance or pantothenic acid auxotrophy.

Moreover, US2005042328A1 describes the intracellular enrichment of γ-glutamylcysteine in a C. utilis strain having reduced glutathione synthetase activity and an elevated gsh1 expression (gsh1 codes for γ-glutamylcysteine synthetase).

Disadvantages of the fungal γGC production systems are the relatively low yields of γ-glutamylcysteine, and also the intracellular accumulation of γ-glutamylcysteine, which would render necessary a disruption of the fungal cells as a possible workup for γ-glutamylcysteine.

Various E. coli strains are described in US20100203592A1 as prokaryotic producers of γ-Glutamylcysteine, all of which have an elevated gshA expression. Moreover, these strains have a normal or elevated glutathione synthetase activity in comparison to the corresponding parent strain. Secretion of γ-glutamylcysteine into the culture medium is possible with these strains in contrast to the fungal systems. The maximum yield with these strains is 262 mg/l, which is insufficient for establishing an economic method.

Microorganism strains do not yet exist, which are capable of overproducing the promising bioactive compound γ-glutamylcysteine and its derivatives bis-γ-glutamylcystine and γ-glutamylcystine, and which allow, moreover, the secretion of these compounds into the cultivation medium on a g/l scale.

### DESCRIPTION OF THE INVENTION

The object of the present invention, therefore, is to provide a microorganism strain which is capable of overproducing γ-glutamylcysteine and its derivatives bis-γ-glutamylcystine and γ-glutamylcystine and which, in addition, enable the extracellular accumulation of these compounds in the cultivation medium.

The object is achieved by a prokaryotic microorganism strain, which can be prepared from a parent strain, and which has a reduced cellular glutathione synthetase activity compared to the parent strain, and has a cellular γ-glutamylcysteine synthetase activity which is more greatly increased than in a strain with similarly reduced cellular glutathione synthetase activity.

The glutathione synthetase activity in the strain according to the invention is preferably so reduced that said activity is at most 50% of the glutathione synthetase activity of a corresponding wild-type strain.

Preference is given to prokaryotic microorganism strains having at most 25% of the glutathione synthetase activity of the wild-type before modification of its glutathione synthetase activity. A reduced glutathione synthetase activity of a strain is especially preferably understood to mean the absence of glutathione synthetase activity in this strain.

The γ-glutamylcysteine synthetase activity in the strain according to the invention is preferably around at least a factor of 5 higher than in a strain with similarly reduced glutathione synthetase activity.

Particular preference is given to microorganism strains according to the invention in which both the glutathione synthetase activity and the γ-glutamylcysteine synthetase activity have been modified as described above.

The degree of DNA identity is determined by the “nucleotide blast” program which can be found on the site http://blast.ncbi.nlm.nih.gov/, which is based on the blastn algorithm. For an alignment of two or more nucleotide sequences, the default parameters were used as algorithm parameters. The default general parameters are: Max target sequences=100; Short queries=“Automatically adjust parameters for short input sequences”; Expect Threshold=10; Word size=28; Automatically adjust parameters for short input sequences=0. The corresponding default scoring parameters are: Match/Mismatch Scores=1, −2; Gap Costs=Linear.

The “protein blast” program, on the site http://blast.ncbi.nlm.nih.gov/, is used for the comparison of protein sequences. This program is based on the blastp algorithm. For an alignment of two or more protein sequences, the default parameters were used as algorithm parameters. The default general parameters are: Max target sequences=100; Short queries=“Automatically adjust parameters for short input sequences”; Expect Threshold=10; Word size=3; Automatically adjust parameters for short input sequences=0. The default scoring parameters are: Template=BLOSUM62; Gap Costs=Existence: 11 Extension: 1; Compositional adjustments=Conditional compositional score template adjustment.

All prokaryotic microorganism strains having the biosynthetic pathway for γ-glutamylcysteine, and which may be cultured by fermentation, are in principle suitable as parent strains for the generation of the microorganism strains according to the invention. Such microorganisms can belong to the domains of the Bacteria (formerly Eubacteria) or Archaea (formerly Archaebacteria).

These organisms are preferably representatives of the phylogenetic group of the bacteria. Particular preference is given to microorgansims of the family of the Enterobacteriaceae, particularly the species Escherichia coli and Pantoea ananatis.

The γ-glutamylcysteine synthetase activity is measured on the basis of γ-glutamylcysteine formation over time, as described in example 9.

The glutathione synthetase activity is determined by the rate of formation of ADP using a pyruvate kinase coupled enzyme test, in which L-glycine and L-γ-glutamyl-L-α-aminobutyrate are reacted as substrates (Kim et al., 2003, J. Biochem. Mol. Biol. 36: 326-331). Suitable parent strains for generating the microorganisms according to the invention are preferably prokaryotic microorganism strains which have the biosynthetic pathway for γ-glutamylcysteine and, in addition, have an elevated L-cysteine biosynthesis capacity compared to a corresponding parent strain. An elevated capacity for L-cysteine biosynthesis is advantageous for the formation of γ-glutamylcysteine, since the provision of sufficient amounts of L-cysteine as γ-glutamylcysteine precursor should not be limiting for a high production of γ-glutamylcysteine. “Elevated L-cysteine biosynthesis capacity” is understood to mean, in accordance with the invention, the capability of a microorganism strain to produce more L-cysteine or its derivatives L-cystine and thiazolidine, than a corresponding wild-type, parent or non-modified strain. This typically manifests as an enrichment of L-cysteine, L-cystine and/or thiazolidine in the medium. An elevated L-cysteine biosynthesis capacity is therefore present, known to those skilled in the art, if during or at the end of a fermentation of the relevant microorganism strain, at least 0.5 g/l of “total cysteine” (L-cysteine+L-cystine+thiazolidine) can be detected in the medium.

Potential parent strains for the generation of microorganisms according to the invention having an elevated capacity for L-cysteine biosynthesis are disclosed, for example, in US20040038352A1, US20090053778, EP1528108A1, EP2345667A2 or EP2138585.

Moreover, genes or variants of theses genes (alleles) are already known from the prior art which leads to their use for overproducing the amino acids L-cysteine and/or the precursors of L-serine or O-acetylserine:
serA alleles as described in EP0620853B1, EP1496111B1 and EP0931833A2:
These serA alleles code for 3-phosphoglycerate dehydrogenases, which are subject to a reduced feedback inhibition by L-serine. In this way, the formation of 3-hydroxypyruvate is largely decoupled from the serine level of the cell, which allows a better mass flow to the L-cysteine.
cysE alleles, as described in WO9715673; Nakamori S. et al., 1998, Env. Microbiol 64: 1607-1611 or Takagi H. et al., FEBS Lett. 452: 323-327:
These cysE alleles code for serine-O-acetyltransferases, which are subject to a reduced feedback inhibition by L-cysteine. In this way, the formation of O-acetylserine and L-cysteine is largely decoupled from the L-cysteine level in the cell.
Efflux genes as described in EP885962A1:
The orf306 described in EP885962A1 has been cited many times over a period of time. The DNA sequence of the gene referred to as orf306 or also orf299, ydeD and eamA is now deposited under the accession number NC_{—}004431 or NC_{—}007779. Orf306 (orf299, eamA or ydeD) codes for an efflux system, which is suitable for eliminating antibiotics and other toxic substances and causes the overproduction of L-cysteine, L-cystine, N-acetylserine and/or thiazolidine derivatives (Ohtsu I. et al., 2010, J. Biol. Chem. 285; 17479-17489; EP885962 and EP1233067B1).
cysB as described in DE19949579C1:
The cysB gene codes for a central regulator of cysteine metabolism and plays a decisive role, inter alia, in the provision of sulfide for cysteine biosynthesis.

A microorganism strain according to the invention preferably also expresses one or more of the abovementioned genes or alleles. Particular preference is given to using one of the alleles described of cysE or serA and also orf306. In this case, the cysE and serA alleles and also orf306 may be expressed individually or in combination in the microorganism strain according to the invention. A microorganism strain according to the invention particularly preferably has genetic modifications which are presented in the examples as inventive, particularly those characterized as inventive in Table 4.

An inventive microorganism strain can be generated using standard techniques of molecular biology.

The cellular activity of the γ-glutamylcysteine synthetase (GshA) may be increased, for example, by increasing the copy number of the gshA gene or a gshA homolog or by using suitable promoters which lead to an increased expression of the gshA gene or a gshA homolog.

The gshA gene of E. coli codes for the γ-glutamylcysteine synthetase (GshA) enzyme. The gshA gene is characterized by SEQ ID No. 1. The GshA gene product (GshA) is characterized by SEQ ID No. 2. In the context of the present invention, gshA homologous genes have a sequence identity greater than 30% in relation to SEQ ID No. 1. gshA homologs particularly preferably have a sequence identity of greater than 70% in relation to SEQ ID No. 1. GshA homologs are proteins having a sequence identity greater than 30% in relation to SEQ ID No. 2. GshA homologs particularly preferably have a sequence identity of greater than 70% in relation to SEQ ID No. 2.

The increase in the copy number of the gshA gene or a gshA homolog in a microorganism can be carried out using methods known to those skilled in the art. Accordingly, gshA or a gshA homolog, for example, can be cloned into plasmid vectors with multiple copy numbers per cell (e.g. pBR322, pBR derivatives, pBluescript, pUC18, pUC19, pACYC184 and pACYC184 derivatives for E. coli) and introduced into the microorganism. Alternatively, the gshA gene or the gshA homolog can be repeatedly integrated into the chromosome of a microorganism strain. The known systems using temperate bacteriophages, integrative plasmids or integration via homologous recombination may be used as integration methods (Hamilton et al., 1989, J. Bacteriol. 171: 4617-4622; Datsenko and Wanner, 2000, P.N.A.S. 97: 6640-6645).

Preference is given to increasing the copy number by cloning of gshA or a gshA homolog into plasmid vectors under the control of a promoter. Particular preference is given to increasing the copy number in E. coli by cloning of gshA or a gshA homolog into a pACYC derivative such as pACYC184-LH (deposited according to the Budapest Treaty in the German Collection of Microorganisms and Cell Cultures, 83124 Braunschweig, InhoffenstraSe 7B on 18.08.1995 under number DSM 10172).

Increasing the copy number is understood to mean preferably an increase of around a factor of 10.

The gshA gene or a gshA homolog is cloned into plasmid vectors, for example, by specific amplification by means of the polymerase chain reaction (PCR) using specific primers which cover the complete gene, and subsequent ligation with plasmid DNA fragments.

The natural promoter and/or operator region of the genes can serve as control region for the expression of plasmid-coded genes.

The expression rates of gshA or a gshA homolog may also be increased by means of other promoters. Relevant promoter systems such as the constitutive GAPDH promoter of the gapA gene in E. Coli or the inducible lac, tac, trc, lambda, ara or tet promoters are known to those skilled in the art (Markides S.C., 1996, Microbiol. Rev. 60: 512-538). Such constructions may be used in a manner known per se in plasmids or chromosomes.

Furthermore, an increased cellular GshA activity may be achieved in that translation start signals, such as the ribosome binding site, or the Shine Dalgarno sequence are present in optimized sequence on the respective construct, or that rare codons according to the “codon usage” are exchanged with frequently used codons.

The cellular GshA activity may also be increased in that a mutation (substitution, insertion or deletion of individual or multiple nucleotides) is introduced into the reading frame of the gshA gene or a gshA homolog, which results in an increase in the specific activity of GshA or a GshA homolog. The exchange of the uncommon start codon TTG of the gshA gene, for example, for the normal start codon ATG leads not only to an increased expression of gshA, but also to an increase of the total cellular activity of GshA in E. coli (Kwak et al., 1998, J. Biochem. Mol. Biol. 31: 254-257). Site-directed mutations of gshA, which on the protein level involves an exchange of alanine 494 for valine or leucine (A494V or A494L) or a substitution of serine at position 495 for threonine (S495T), also leads to an increased cellular GshA activity in E. coli (Kwak et al., 1998, J. Biochem. Mol. Biol. 31: 254-257). Such alleles are preferably gshA homologs in accordance with the invention.

Methods for reducing glutathione synthetase activity in a microorganism strain are also known from the prior art. The cellular glutathione synthetase activity may be reduced, for example, in that a mutation (substitution, insertion or deletion of individual or multiple nucleotides) is introduced into the reading frame of the gshB gene or a gshB homolog, which leads to a reduction in the specific activity of GshB or a GshB homolog. Methods for generating such gshB alleles are known to those skilled in the art. Alleles of the gshB gene can be prepared, for example, by non-specific or directed mutagenesis using the DNA of the gshB wild-type gene as starting material. Examples of such alleles, which code for GshB variants having a reduced GshB activity compared to the wild-type enzyme, are decribed, for example, in Kato et al. (1988, J. Biol. Chem. 263: 11646-11651).

Non-specific mutations within the gshB gene or the promoter region of the gshB gene can be generated, for example, by chemical agents such as, inter alia, nitrosoguanidine, ethylmethanesulfonic acid and/or by physical methods and/or by PCR reactions carried out under certain conditions. Methods for introducing mutations at specific positions within a DNA fragment are known. For example, one or more bases in a DNA fragment, which comprises the gshB gene and its promoter region, can be exchanged by means of PCR using suitable oligonucleotides as primers. Additionally, it is possible to prepare the whole gshB gene or a new gshB allele by means of gene synthesis.

gshB alleles are generally initially generated in vitro and subsequently inserted into the chromosome of the cell, whereby the gshB wild-type gene originally present is replaced and thus a gshB mutant strain is generated. gshB alleles can be inserted into the chromosome of a host cell instead of the gshB wild-type gene by known standard methods. This can be carried out, for example, by the method described in Link et al. (1997, J. Bacteriol. 179: 6228-37) for introducing chromosomal mutations into a gene via the mechanism of homologous recombination. The chromosomal deletion of the whole gshB gene or a part thereof is possible, for example, by means of the A-Red recombinase system according to the methods described by Datsenko and Wanner (2000, Proc. Natl. Acad. Sci. USA. 97: 6640-5). gshB alleles may also be transferred from a strain having a gshB mutation to a gshB wild-type strain via a transduction by means of P1 phages or conjugation, wherein the gshB wild-type gene in the chromosome is replaced by the corresponding gshB allele.

Moreover, the glutathione synthetase activity of a cell can also be reduced in that at least one element required for the expression regulation (e.g. promoter, enhancer, ribosomal binding site) is mutated by substitution, insertion or deletion of individual or multiple nucleotides.

The gshB gene of E. coli codes for the glutathione synthetase enzyme. The gshB gene is characterized by SEQ ID No. 3. The GshB gene product (GshB) is characterized by SEQ ID No. 4.

In the context of the present invention, gshB homologous genes have a sequence identity greater than 30% to SEQ ID No. 3. Particular preference is given to a sequence identity of greater than 70% to SEQ ID No. 3. GshB homologs are proteins having a sequence identity greater than 30% to SEQ ID No. 4. GshB homologs particularly preferably have a sequence identity of greater than 70% to SEQ ID No. 4.

The invention furthermore relates to a method for overproducing γ-glutamylcysteine (γGC) and the derivatives of this dipeptide, γ-glutamylcystine and bis-γ-glutamylcystine, wherein a microorganism strain according to the invention is cultured in a fermentation medium, the cells are removed from the medium and the desired products are purified from the culture medium.

The microorganisms required for the method according to the invention are cultured (fermented) on an industrial scale in a bioreactor (fermenter) by customary fermentation methods known to those skilled in the art.

The fermentation is preferably carried out in a conventional bioreactor, for example, a stirred tank, a bubble column fermenter or an airlift fermenter. Particular preference is given to a stirred tank fermenter. Industrial scale in this case is understood to mean a fermenter size of at least 2 l. Preference is given to fermenters having a volume of greater than 5 l, particularly preferably fermenters having a volume of >50 l.

The cells for γ-glutamylcysteine production are cultured under aerobic growth conditions, where the oxygen content during the fermentation is adjusted to at most 50% saturation. The oxygen saturation in the culture is regulated automatically via the gas supply and the stirrer speed.

Preference is given to sugar, sugar alcohols, organic acids or sugar-containing plant hydrolysates as carbon sources. In the method according to the invention, particular preference is given to using glucose, fructose, lactose, glycerol or mixtures comprising two or more of these compounds as carbon sources.

The carbon source is preferably added to the culture such that the content of the carbon source in the fermenter during the production phase does not exceed 10 g/l. A maximum concentration of 2 g/l is preferred.

In the method according to the invention, preference is given to using ammonia, ammonium salts or protein hydrolysates as nitrogen sources. When using ammonia as the correction means for stabilizing the pH, this nitrogen source is regularly added during the fermentation.

Salts of the elements phosphorus, chlorine, sodium, magnesium, nitrogen, potassium, calcium, iron and in traces, i.e. in μM concentrations, salts of the elements molybdenum, boron, cobalt, manganese, zinc and nickel, may be added as further media supplements.

In addition, organic acids (e.g. acetate, citrate), amino acids (e.g. L-glutamic acid, L-cysteine) and vitamins (e.g. B1, B6) may be added to the medium.

L-Glutamic acid can in this case either be used directly as the acid or in the form of one of its salts, e.g. potassium or sodium glutamate, individually or as a mixture. Preference is given to using potassium glutamate.

The complex nutrient sources used may be e.g. yeast extract, corn steep liquor, soy meal or malt extract.

The incubation temperature for mesophilic microorganisms such as E. coli is preferably 15-45° C., particularly preferably 30-37° C.

The production phase of the inventive fermentation method starts at the time point at which γ-glutamylcysteine, bis-γ-glutamylcystine or γ-glutamylcystine can first be detected in the culture broth. This phase typically begins ca. 8-12 h after inoculation of the production fermenter with a preculture.

Microorganisms, which are fermented according to the method described in a batch or fedbatch process, secrete γ-glutamylcysteine and the compounds γ-glutamylcystine and bis-γ-glutamylcystine derived therefrom, into the fermentation medium at high efficiency after a growth phase of a period of at least 48 h.

In the context of the invention, the overproduction of γ-glutamylcysteine is preferably understood to mean the capability of a microorganism strain to produce more γ-glutamylcysteine or its derivatives γ-glutamylcystine and bis-γ-glutamylcystine than a corresponding wild-type, parent or unmodified strain. This typically manifests as an enrichment of γ-glutamylcysteine, γ-glutamylcystine and/or bis-γ-glutamylcystine in the medium. An overproduction of γ-glutamylcysteine is present, therefore, if at the end of a fermentation of the relevant microorganism strain, yields of at least 0.2 g/l of “total γ-glutamylcysteine” (γ-glutamylcysteine+γ-glutamylcystine+bis-γ-glutamylcystine) can be detected in the medium. Yields of “total γ-glutamylcysteine” in the medium are preferably in the range of 3 to 23 g/l, as can be achieved, for example, with the inventive microorganisms listed in Tables 3 and 4. Yields in the range of 10 to 23 g/l of “total γ-glutamylcysteine” in the medium are particularly preferred with respect to establishing an economic method.

### BRIEF DESCRIPTION OF THE DRAWINGS

FIG. 1 shows a restriction and functional map of plasmid pACYC184-LH.

FIG. 2 shows a restriction and functional map of pgshAₚ-gShA^{TTG}.

FIG. 3 shows a restriction and functional map of ptufB.

FIG. 4 shows a restriction and functional map of ptufBₚ-gshA^{ATG}.

FIG. 5 shows a restriction and functional map of pgshA^{ATG}-serA2040.

FIG. 6 shows a restriction and functional map of pgshA^{ATG}-cysE14.

FIG. 7 shows a restriction and functional map of pgshA^{ATG}-cysE14-serA2040.

FIG. 8 shows a restriction and functional map of pgshA^{ATG}-serA2040-orf306.

FIG. 9 shows a restriction and functional map of pgshA^{ATG}-cysE14-orf306.

FIG. 10 shows a restriction and functional map of pgshA^{ATG}-cysE14-serA2040-orf306.

FIG. 11 shows HPLC chromatograms of A) an untreated (oxidized) sample and B) a (reduced) sample treated with DTT.

The following examples serve to further illustrate the invention:

### Example 1

### Deletion of the Gene gshB (Gutathione Synthetase Inactivation) in the E. coli Strain W3110 (ATCC27325)

The gene gshB, which codes for the enzyme glutathione synthetase (GshB) in E. coli, was deleted in the E. coli strain W3110 (ATCC27325) according to the “A-Red Method” developed by Datsenko and Wanner (Datsenko and Wanner, 2000, P.N.A.S. 97: 6640-6645). A DNA fragment, which codes for the kanamycin resistence marker (kanR), was amplified using the primers gshB-del-for (SEQ ID No. 5) and gshB-del-rev (SEQ ID No. 6).

The primer gshB-del-for codes for a sequence consisting of 30 nucleotides, which is homologous to the 5′-end of the gshB gene, and a sequence comprising 20 nucleotides, which is complementary to a DNA sequence which codes one of the two FRT sites (FLP recognition target) on the plasmid pKD13 (Coli Genetic Stock Center (CGSC) No. 7633). The primer gshB-del-rev codes for a sequence consisting of 30 nucleotides, which is homologous to the 3′-end of the gshB gene, and a sequence comprising 20 nucleotides, which is complementary to a DNA sequence which codes the second FRT site on the plasmid pKD13.

The amplified PCR product was transformed by means of electroporation into the Escherichia coli strain W3110 (ATCC27325), as described in example 2 of U.S. Pat. No. 5,972,663A. The transformed, gshB-deficient cells were selected on LB agar plates containing 50 mg/l kanamycin. The marker gene (kanamycin resistance, kanR) was removed with the FLP recombinase enzyme, which has been coded on the plasmid pCP20 (CGSC No. 7629). Due to a temperature-sensitive “origin of replication” (ori), the plasmid pCP20 can be removed again after transformation by incubating the E. coli cells at 43° C. The E. coli gshB deletion strain generated in this manner bears the designation W3110AgshB.

### Example 2

### Amplification of the gshA Gene with its Own Promoter

The promoter sequence of the gshA gene in E. coli is known (Watanabe et al., 1986, Nucleic Acids Res. 14: 4393-4400). A DNA fragment, which codes for the gshA gene and the gshA promoter (gshAₚ) of E. coli, was amplified by means of PCR. The DNA fragment, which codes for the promoter sequence of the gshA gene, was amplified with the primers gshAₚ-gshA-for (SEQ ID No. 7) and gshAₚ-gshA-rev (SEQ ID No. 8). Chromosomal DNA of the E. coli strain W3110 (ATCC 27325) served as template for the PCR reaction.

The approximately 1.9 kb sized PCR fragment was purified by agarose gel electrophoresis and isolated from the agarose gel using the “QIAquick Gel Extraction Kit” (Qiagen GmbH, Hilden, D) according to the manufacturer's instructions. The purified PCR fragment was then digested with the restriction enzyme XbaI and stored at −20° C.

### Example 3

### Amplification and Mutagenesis of the gshA Gene

A. Amplification of the gshA gene

The gshA gene of E. coli was amplified by means of PCR. Chromosomal DNA of the E. coli strain W3110 (ATCC 27325) served as template for the PCR reaction. A Taq polymerase (Qiagen GmbH) was used for the amplification, which attaches an additional adenine at the respective 3′-end of the PCR product. In the course of the amplification of gshA, the uncommon start codon TTG was replaced by start codon ATG by using a suitable primer. The oligonucleotides gshA-OE-for (SEQ ID NO. 9) and gshA-OE-rev (SEQ ID NO. 10) served as specific primers.

The resulting 1.6 kb sized DNA fragment was purified by agarose gel electrophoresis and isolated from the agarose gel using the “QIAquick Gel Extraction Kit” (Qiagen GmbH). The ligation of the amplified and purified PCR product with the vector pCR2.1-TOPO (Life Technologies, Life Technologies GmbH, Darmstadt, D) was carried out by “TA cloning” using the “TOPO® TA Cloning® Kit with PCR®2.1 TOPO®”, according to the manufacturer's instructions (Life Technologies GmbH).

The ligation mixture was transformed into “DH5α™-T1R E. coli cells” (Life Technologies), propagated in these cells and, following plasmid isolation, the DNA sequence of the plasmids was verified by means of sequencing. The resulting construct with the desired sequence bears the designation pCR2.1-gshA.

B. Mutagenesis of the gshA Gene

For recloning of gshA, the two interfering restriction sites EcoRI and BglII in the gshA gene were initially removed by means of directed mutagensis. The mutagenesis reactions were carried out using “GeneTailor™ Site-Directed Mutagenesis System” (Life Technologies GmbH) according to the manufacturer's instructions. For the first mutagenesis, the plasmid pCR2.1-gshA served as template. The mutagenesis primers gshA-EcoRImut-for (SEQ ID No. 11) and gshA-EcoRImut-rev (SEQ ID No. 12) were used to remove the EcoRI cleavage site in the gshA gene. Subsequent to the mutagenesis conducted on the EcoRI cleavage site in the gshA gene, a part of the mutagenesis reaction was again transformed into “DH5α™-T1R E. coli cells” from Life Technologies and propagated in these cells. Following plasmid preparation, the DNA sequence of the isolated plasmids was verified by sequencing. The resulting construct with correct DNA sequence without EcoRI cleavage site within the gshA gene bears the designation pCR2.1-gshA_mut1.

A similar procedure for removing the BglII cleavage site in the gshA gene of pCR2.1-gshA_mut1 was carried out, only that the mutagenesis primers gshA-BglIImut-for (SEQ ID No. 13) and gshA-BglIImut-rev (SEQ ID No. 14) were used for the mutagenesis reaction.

A part of the second mutagenesis reaction was again transformed into “DH5α(m-T1R E. coli cells” (Life Technologies GmbH), propagated in these cells and, following plasmid preparation once again, the DNA sequence of the isolated plasmids was verified by means of sequencing. The resulting construct with correct DNA sequence without EcoRI and Bg1II cleavage sites within the gshA gene bears the designation pCR2.1-gshA mutt.

### Example 4

### Amplification of the DNA Fragments Coding for the tufB promoter (ₚtufB)

For effective transcription (overexpression) of the gshA gene, the activating region of the tRNA-tufB operon was used (Lee et al., 1981, Cell 25: 251-258). This operon in E. coli codes for the structural genes thrU, tyrU, glyT and thrT, and also for the “Protein Chain Elongation Factor EF-Tu” (TufB) protein. The desired promoter sequence was amplified with the oligonucleotides tufBₚ-for (SEQ ID No. 15) and tufBₚ-rev (SEQ ID No. 16). Genomic DNA of the strain E. coli W3110 (ATCC27325) again served as template. The DNA fragment obtained was purified by agarose gel electrophoresis and isolated from the agarose gel using the “QIAquick Gel Extraction Kit” (Qiagen GmbH). The purified PCR fragment was then digested with the restriction enzyme XbaI and stored at −20° C.

### Example 5

### Construction of the Plasmids for gshA Overexpression (gshA Plasmids)

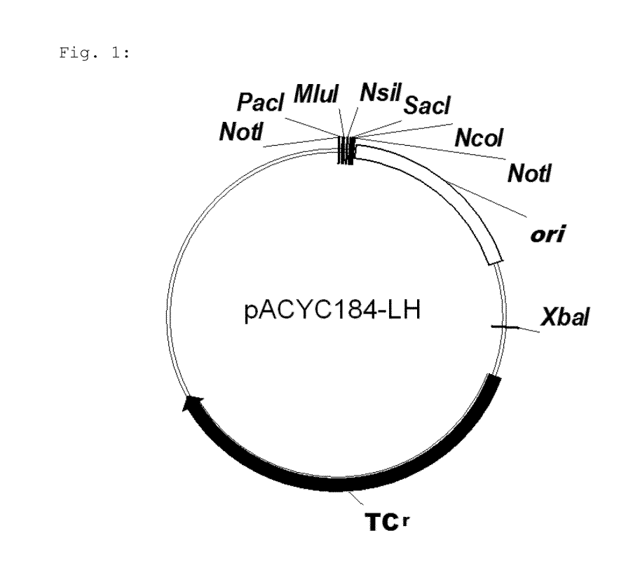

The plasmid pACYC184-LH was used as base plasmid for the construction of the plasmids according to the invention. A restriction and function map of plasmid pACYC184-LH is shown in FIG. 1. Besides a XbaI recognition sequence, the plasmid pACYC184-LH codes also for a polylinker region with the following restriction sites:

NotI-NcoI-Scal-NsiI-MluI-Pad-NotI

A. Cloning of the gshA Gene with its Own Promoter

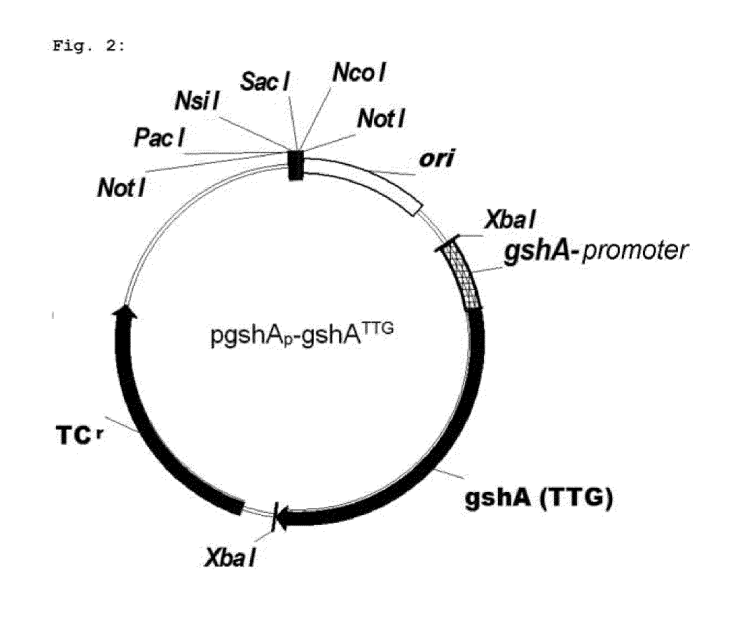

Initially, the PCR product digested with XbaI, described in example 2, and coding for the gshA gene with its own promoter and the uncommon start codon TTG, was cloned into the XbaI cleavage site of pACYC184-LH. The ligation mixture was transformed into “DH5α™-T1R E. coli cells” (Life Technologies GmbH), propagated in these cells and the DNA sequence of the isolated plasmids was verified by means of sequencing. The resulting construct bears the designation pgshAₚ-gshA^{TTG} (see FIG. 2).

B. Cloning of the tufB Promoter

The PCR product digested with XbaI, described in example 4, and coding for the tufB promoter (tufBₚ), was also cloned into the XbaI cleavage site of pACYC184-LH. The ligation mixture was transformed into “DH5α™-T1R E. coli cells” (Life Technologies GmbH), propagated in these cells and the DNA sequence of the isolated plasmids was verified by means of sequencing. The resulting construct bears the designation pACYC184-tufBₚ.

C. Optimizing of the tufB Promoter

The plasmid pACYC184-tufBₚ served as template for a total of optimizations (mutageneses), which were conducted on the DNA sequence of the native tufB promoter. By means of the “GeneTailor™ Site-Directed Mutagenesis System” (Life Technologies GmbH), the ribosomal binding site (RBS), the −10 region and the −35 region of the tufB promoter, were optimized so that they ultimately coded for the corresponding consensus sequences of these prokaryotic promoter elements (see Table 1).

TABLE 1
Comparison of the DNA sequences of the RBS, −10
region and the −35 region of the tufB promoter
with the corresponding consensus sequences. The
mutagenized nucleotides are highlighted in bold.

| | in the tufB | |
|---|---|---|
| Element | promoter | Consensus sequence |
| RBS | 5′-ACTTGG-3′ | 5′-AGGAGG-3′ |
| −10 region | 5′-TAGAAT-3′ | 5′-TATAAT-3′ |
| −35 region | 5′-TTGCAT-3′ | 5′-TTGACA-3′ |

For optimizing the RBS by site-directed mutagenesis of the tufB promoter on the plasmid pACYC184-tufBₚ, the mutagenesis primers tufBₚ-RBSₒₚₜ-for (SEQ ID No. 17) and tufBₚ-RBSₒₚₜ-rev (SEQ ID No. 18) were used.

Subsequent to the mutagenesis reaction conducted on the RBS of the tufB promoter with the “GeneTailor™ Site-Directed Mutagenesis System” (Life Technologies), a part of the mutagenesis reaction was transformed into “DH5α™-T1R E. coli cells” (Life Technologies GmbH), propogated in these cells and, after plasmid preparation, the DNA sequence of the isolated plasmids was verified by means of sequencing. The resulting construct with correct DNA sequence and optimized RBS sequence bears the designation pACYC184-tufBₚ—RBSₒₚₜ.

For optimizing (mutagenesis) of the −10 region based on pACYC184-tufBₚ-RBSₒₚₜ, a similar process was used, only that the primers tufBₚ-10ₒₚₜ-for (SEQ ID No. 19) and tufBₚ-10ₒₚₜ-rev (SEQ ID No. 20) were used for the mutagenesis.

The plasmid formed and checked by means of sequencing with optimized RBS and −10 region bears the designation pACYC184-tufBₚ (RBS-10)ₒₚₜ.

The plasmid pACYC184-tufBₚ(RBS-10)_{Opt} again served as template for the mutagenesis of the -35 region, which was carried out with the primers tufBₚ-35ₒₚₜ-for (SEQ ID No. 21) and tufBₚ-35ₒₚₜ-rev (SEQ ID No. 22).

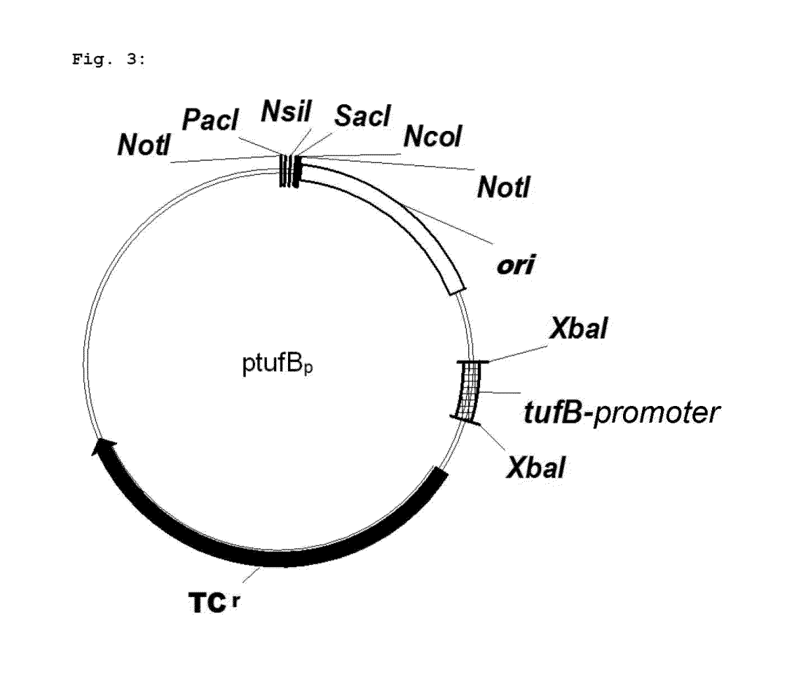

The plasmid formed and checked by means of sequencing with optimized RBS, −10 region and −35 region bears the designation ptufBₚ. A restriction and functional map of the resulting plasmid ptufBₚ is shown in FIG. 3.

D. Cloning of the gshA Gene with Optimized Start Codon behind the Optimized tufB Promoter in ptufBₚ

The mutagenized gshA gene with the start codon ATG, described in example 3, was removed from the plasmid pCR2.1-gshA_mutt by a restriction digestion with the enzymes EcoRI and Bg1II and recloned into the vector ptufBₚ cut with EcoRI and Bg1II.

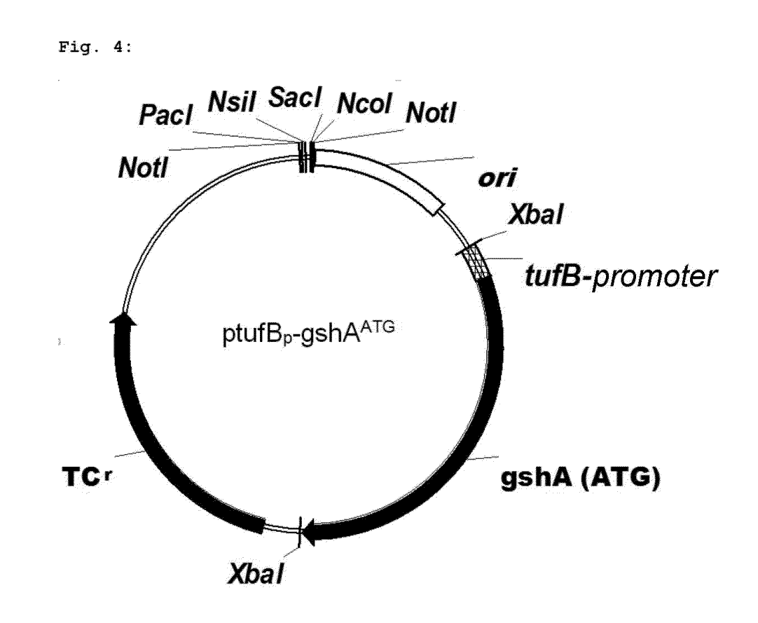

The resulting construct ptufBₚ-gshA^{ATG} (see FIG. 4) was transformed into “DH5α™-T1R E. coli cells” (Life Technologies GmbH), propagated in these cells and the DNA sequence of the isolated plasmids was verified by means of sequencing.

### Example 6

### Extending the gshA expression plasmid ptufBₚ-gshA^{ATG} with serA and/or cysE alleles and also orf306 (=orf299, ydeD or eamA)

In order to investigate the influence of increased L-cysteine biosynthesis on the γ-glutamylcysteine production, the plasmid ptufBₚ-gshA^{ATG} was extended with serA and/or cysE alleles and also the orf306 (=orf299, eamA or ydeD).

a. Extending Plasmid ptufBₚ-gshA^{ATG} with serA Alleles

To extend the plasmid ptufBₚ-gshA^{ATG} with a specific serA allele, the serA gene was initially amplified with its own promoter via a PCR. Chromosomal DNA of the E. coli strain W3110 (ATCC 27325) served as template for the PCR reaction. The oligonucleotides serA-NcoI-for (SEQ ID. No. 23) and serA-SacI-rev (SEQ ID No. 24) were used for the PCR reaction.

The amplified serA gene, including its own promoter, was then digested with the enzymes NcoI and SacI and, following purification on agarose gel, was ligated into the ptufBₚ-gshA^{ATG} vector cut with NcoI and SacI.

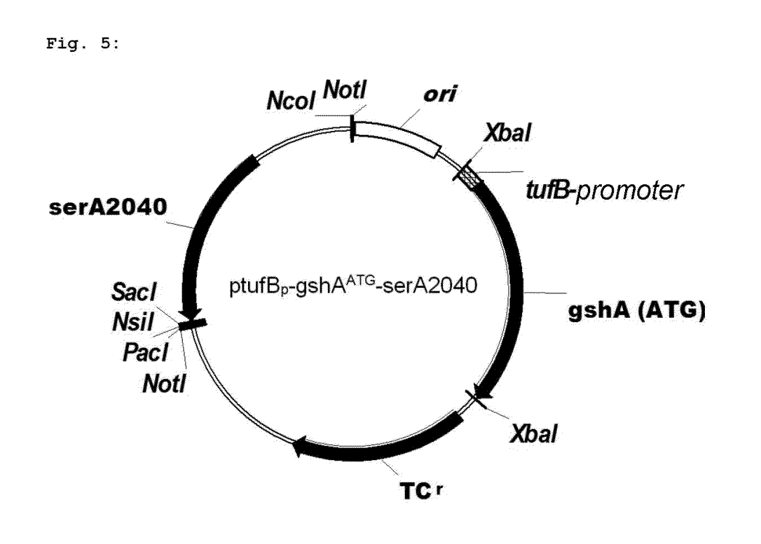

The ligation mixture was transformed into “DH5α™-T1R E. coli cells” (Life Technologies GmbH), propagated in these cells and the DNA sequence of the isolated plasmids was verified by means of sequencing. The gshA plasmid arising from this cloning with serA under the control of the native promoter was designated ptufBₚ-gshA^{ATG}-serA. For generating various serA alleles, which code for SerA variants which are feedback-resistant to L-serine, the procedure as in example of EP1496111B1 was carried out, except that the plasmid ptufBₚ-gshA^{ATG}-serA served as template for changing the codons 349 and/or 372 in place of pFL209. The gshA plasmids arising from the mutagenesis with different serA alleles were designated ptufBₚ-gshA^{ATG}-serA . . . , where . . . corresponds to the respective serA allele number (see e.g. FIG. 5 for ptufBₚ-gshA^{ATG}-serA2040).

B. Extending Plasmid ptufBₚ-gshA^{ATG} with a cysE Allele

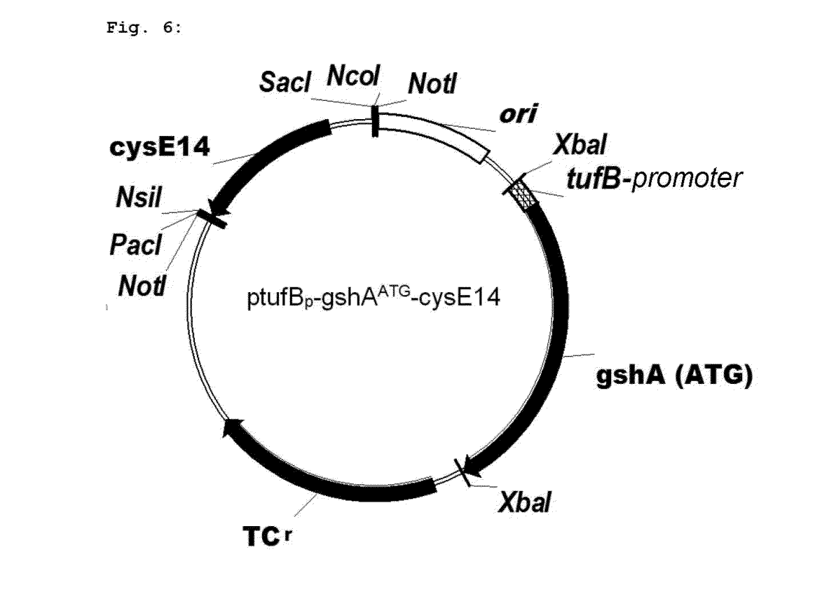

To extend the plasmid ptufBₚ-gshA^{ATG} with a specific cysE allele, one of the pACYC184/cysE . . . plasmids disclosed in Example 6 (Table No. 7) of WO9715673 was initially digested with NsiI and NcoI. The respective approx. 1.0 kb sized fragment, which codes for a specific cysE allele under control of the native cysE promoter, was purified on an agarose gel and subsequently ligated into the vector ptufBₚ-gshA^{ATG} linearized with NsiI and NcoI. The gshA plasmids arising from this cloning with different cysE alleles were designated ptufBₚ-gshA^{ATG}-cysE . . . , where . . . corresponds to the respective cysE allele number (see e.g. FIG. 6 for ptufBₚ-gshA^{ATG}-cysE14).

C. Extending Plasmid ptufBₚ-gshA^{ATG}-serA . . . With a cysE Allele

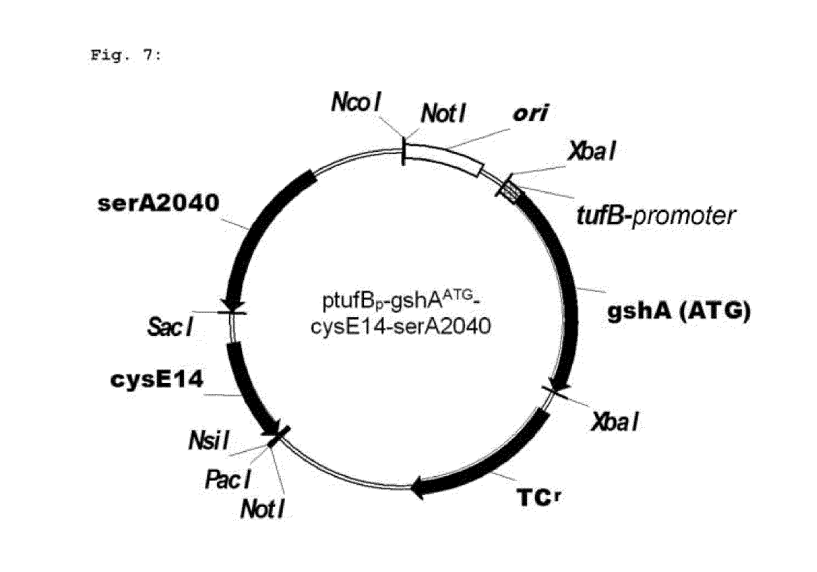

To extend the plasmid ptufBₚ-gshA^{ATG}-serA . . . with a specific cysE allele, one of the plasmids pACYC184/cysE . . . disclosed in Example 6 (Table No. 7) of WO9715673 was initially digested with SacI and NsiI. The approx. 1.0 kb sized fragment was purified on an agarose gel and subsequently ligated into a vector ptufBₚ-gshA^{ATG}-serA . . . linearized with SacI and NsiI. The gshA plasmids arising from this cloning with different cysE and serA alleles were designated ptufBₚ-gshA^{ATG}-cysE . . . where . . . corresponds to the respective cysE or serA allele number (see e.g. FIG. 7 for ptufBₚ-gshA^{ATG}-cysE14-serA2040).

D. Extending the Plasmids ptufBₚ-gshA^{ATG}-serA . . . , ptufBₚ-gshA^{ATG} cysE . . . And ptufBₚ-gshA^{ATG}-cysE . . . -serA . . . With the Orf306

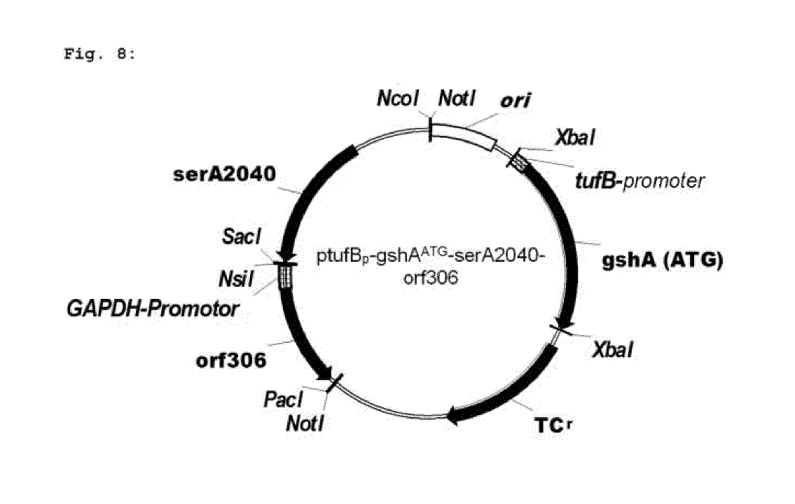

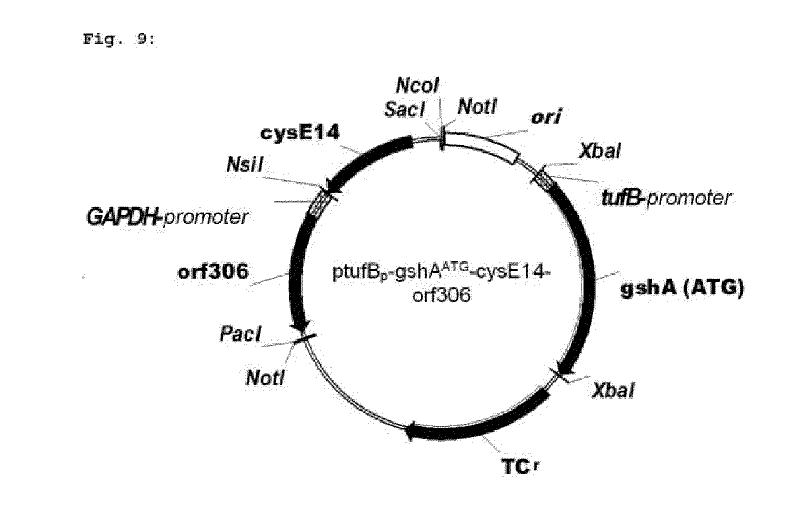

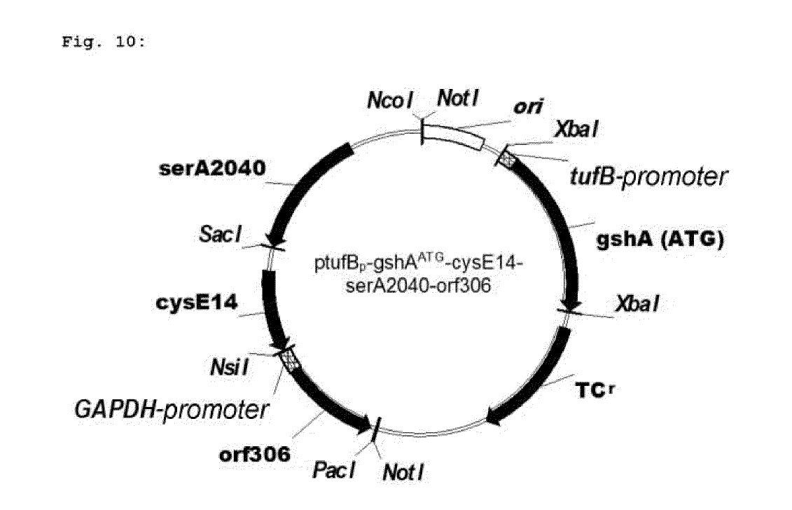

To extend the corresponding plasmids ptufBₚ-gshA^{ATG}, ptufBₚ-gshA^{ATG}-serA . . . , ptufBₚ-gshA^{ATG}-cysE . . . and ptufBₚ-gshA^{ATG}-cysE . . . -serA . . . with the orf306, the plasmid pACYC184/cysEIV-GAPDH-ORF306 disclosed in example 2 of EP885962B1 was initially digested with NsiI and PacI. The approx. 1.2 kb sized fragment, which codes for the 0-acetylserine/cysteine exporter (EamA, YdeD), was purified on agarose gel and subsequently ligated into one of the vectors ptufBₚ-ptufBₚ-gshA^{ATG}-serA . . . , gshA^{ATG}-cysE . . . or ptufBₚ-gshA^{ATG}-cysE . . . -serA . . . linearized with NsiI and PacI. The gshA plasmids arising from this cloning with orf306 under control of the GAPDH promoter were designated ptufBₚ-gshA^{ATG}-serA . . . -orf306, ptufBₚ-gshA^{ATG}-cysE . . . -orf306 and ptufBₚ-gshA^{ATG}-cysE . . . serA . . . -orf306, where . . . corresponds to the respective cysE or sera allele number (see e.g. FIG. 8 for ptufBₚ-gshA^{ATG}-serA2040-orf306, FIG. 9 for ptufBₚ-gshA^{ATG}-cysE14-orf306 and FIG. 10 for ptufBₚ-gshA^{ATG}-cysE14-serA2040-orf306).

### Example 7

### Transformation of the E. coli strain W3110 (ATCC 27325) and W3110AgshB

The gshA expression plasmids pgshAₚ-gshA^{TTG}, ptufBₚ-gshA^{ATG}, ptufBₚ-gshA^{ATG}-serA2040, ptufBₚ-gshA^{ATG}-cysE14, ptufBₚ-gshA^{ATG}-cysE14-serA2040, ptufBₚ-gshA^{ATG}-serA2040-orf306, ptufBₚ-gshA^{ATG}-cysE14-orf306 and ptufBₚ-gshA^{ATG}-cysE14-serA2040-orf306, and plasmid pACYC184-LH as negative control, were transformed into the E. coli strains W3110 (ATCC27325) and W3110ΔgshB by means of electroporation, as already described in example 1. The selection of plasmid-bearing cells was carried out on LB agar plates comprising 15 mg/L tetracycline.

### Example 8

### Analysis of γ-Glutamylcysteine and the Derivatives

The term “total γ-glutamylcysteine” includes γ-glutamylcysteine and the oxidation products bis-γ-glutamylcystine and γ-glutamylcystine formed therefrom, which are formed during the fermentation and accumulate in the culture supernatant. The concentrations of “total γ-glutamylcysteine” were determined by HPLC analysis.

a. Pretreatment of the Samples

To measure the fermenter samples, the microorganisms were initially removed by a centrifugation step and a subsequent sterile filtration of the fermentation broth.

For the exact determination of the “total γ-glutamylcysteine concentration”, the γ-glutamylcysteine derivatives in the sample to be measured were reduced with a molar excess of dithiothreitol (DTT) at room temperature (22° C.) for 1 hour. Since the reducing effect of DTT only occurs fully at neutral to alkaline pH, the pH of the sample was adjusted beforehand to a pH of >7.0 with concentrated aqueous potassium hydroxide solution (KOH) if necessary.

### B. HPLC Analysis

After preparation of the corresponding dilutions with demineralized water, the HPLC analysis was carried out with a Synergi 4 μm Hydro-RP 250×4.6 mm column (Phenomenex Ltd., Aschaffenburg, D) at 20° C. 0.5% (v/v) phosphoric acid (A) and acetonitrile (B) was used as eluent.

The following HPLC method was used for the separation of γ-glutamylcysteine from a cell-free and reduced substance mixture.
Flow rate=0.5 ml/min
Detection at 200 nm using a diode array detector (DAD)
The separation was performed isocratically at 100% A.
After each run the column was cleaned by a rinse step with 100% B.

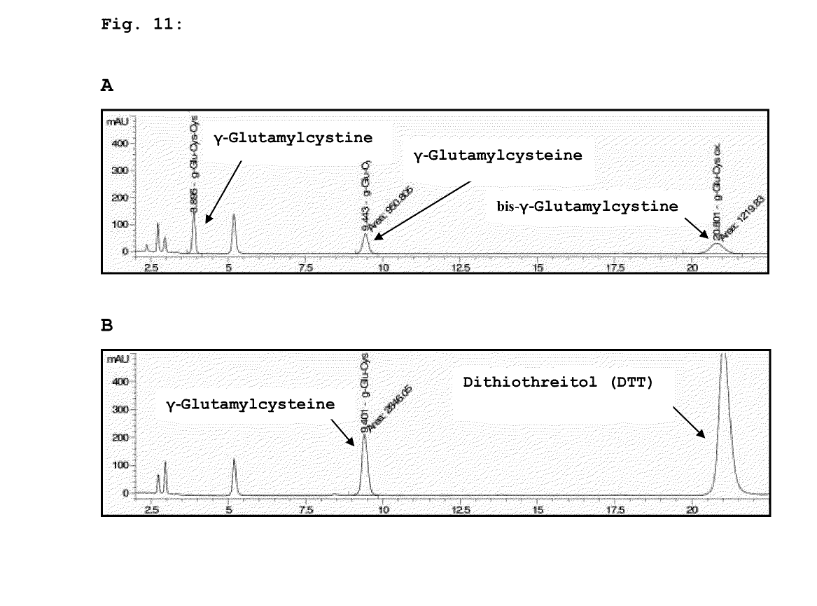

For the determination of the retention time of γ-glutamylcysteine and of the final concentration in the samples to be measured, reduced γ-glutamylcysteine from Sigma Aldrich GmbH (Steinheim, D) was used as reference substance. The concentration of “total γ-glutamylcysteine” was calculated from the peak areas in the chromatogram (see FIG. 11).

### Example 9

### Determination of the γ-glutamylcysteine Synthetase Activity

For the determination of the γ-glutamylcysteine synthetase enzyme activity, 30 ml of SM1 medium (12 g/l K₂HPO₄, 3 g/l KH₂PO₄, 5 g/l (NH₄)₂SO₄, 0.3 g/l MgSO₄×7 H₂O, 0.015 g/l CaCl₂ ×2 H₂O, 0.002 g/l FeSO₄×7 H₂O, 1 g/l Na citrate×2H₂O, 0.1 g/l NaCl; 1 ml/l trace element solution consisting of 0.15 g/l Na₂MoO₄×2H₂O, 2.5 g/l H₃BO₃, 0.7 g/l CoCl₂ ×6 H₂O, 0.25 g/l CuSO₄×5 H₂O, 1.6 g/l MnCl₂×4 H₂O, 0.3 g/l ZnSO₄×7 H₂O), which had been supplemented with 15 g/l glucose, 5 mg/l vitamin B₁ and 15 mg/l tetracycline, were inoculated with a 2 ml overnight culture of the strains listed in Table 2. Based on an initial optical density at 600 nm (OD₆₀₀) of 0.025, the whole 30 ml batch was incubated at 30° C. and 135 rpm for a further 16 hours.

The cells were subsequently harvested by centrifugation, washed and resuspended in 2 ml of buffer (100 mM Tris-HCl pH 8.2). The cells were disrupted by means of a French Press (Spectronic Instruments, Inc. Rochester, N.Y., USA) at a pressure of 124 106 kPa. The crude extracts were clarified by centrifugation at 30 000 g and the γ-glutamylcysteine synthetase activity measured based on the formation of γ-glutamylcysteine over time. The reaction mixture underlying the γ-glutamylcysteine formation assay consists of: 100 mM Tris-HCl pH 8.2; 10 mM potassium glutamate; 10 mM L-cysteine; 20 mM MgCl₂; 150 mM KCl; 20 mM ATP and 0.25 to 4 mg/ml crude extract protein. The ATP-dependent formation of γ-glutamylcysteine from L-cysteine and potassium glutamate was initiated by addition of crude extract protein. Aliquots (20 μl to 50 μl) of the reaction mixture were removed over a period of at least 2 hours. The γ-glutamylcysteine synthetase activity was then inactivated in the samples (aliquots) at 70° C. for 5 min and the content of γ-glutamylcysteine formed was determined by HPLC (see example 8). A unit of γ-glutamylcysteine synthetase activity corresponds to that amount of enzyme which forms one μmol of γ-glutamylcysteine per minute at 25° C.

TABLE 2
γ-Glutamylcysteine synthetase activity of the investigated
gshB deletion strains with plasmid-coded gshA overexpression.

| E. coli strain | mU/mg protein crude extract |
|---|---|
| W3110ΔgshB/pACYC184-LH | 1.8 ± 0.1 |
| W3110ΔgshB/pgshAₚ-gshA^{TTG} (*) | 9.1 ± 1.9 |
| W3110ΔgshB/ptufBₚ-gshA^{ATG} (*) | 143.2 ± 22.9 |

((*) inventive strain)

### Example 10

### γ-Glutamylcysteine Production (Fermentation)

a. Preculture 1 (Shaking Flask):

100 ml of LB medium with 15 mg/l tetracycline in a 1 l Erlenmeyer flask with chicanes were inoculated with the E. coli strains from an agar culture listed in Tables 3 and 4 and incubated at 32° C. and 130 rpm on a shaker for seven hours.

### B. Preculture 2 (Prefermenter):

A portion of the respective preculture 1 was used to inoculate a fermenter filled with fermentation medium of the type Sixfors (Infors A G, Bottmingen, C H), such that an optical density of ca. 0.01 (measured at 600 nm) was present in the fermenter at the start. The fermenter employed had a total volume of 1 l and an initial working volume of 0.7 l.

The fermentation medium comprised the following constituents: 3 g/l (NH₄)₂SO₄, 1.7 g/l KH₂PO₄, 0.25 g/l NaCl, 0.6 g/l MgSO₄×7 H₂O, 0.03 g/l CaCl₂×2 H₂O, 0.15 g/l FeSO₄×7 H₂O, 1 g/l Na₃citrate×2 H₂O, 5 g/l Cornsteep Dry (CSD) and 3 ml/l trace element solution (consisting of 2.5 g/l H₃BO₃, 0.7 g/l CoCl₂×6 H₂O, 0.25 g/l CuSO₄×5 H₂O, 1.6 g/l MnCl₂×4 H₂O, 0.3 g/l ZnSO₄×7 H₂O, 0.15 g/l Na₂MoO₄×2 H₂O). After sterilization of this basal medium, the following constituents were added under sterile conditions: 40 g/l glucose, 0.018 g/l vitamin B1, 0.09 g/l vitamin B6 and 15 mg/l tetracycline.

The pH in the fermenter was adjusted to 7.0 by addition of a 25% NH₄OH solution. During the fermentation, the pH was maintained at a value of 7.0 by automatic correction with 25% NH₄OH. The cultures were stirred at the start at 400 rpm and were flushed with sterilized compressed air via a sterile filter at a gas flow rate of 0.7 volume per volume per minute (vvm). Under these initial conditions, the oxygen probe had been calibrated to 100% saturation prior to inoculation. The target value for the O₂ saturation during the fermentation was set to 50%. On dropping below the target value for O₂ saturation, a regulation cascade was initiated in order to restore the O₂ saturation to the target value. In this case, the gas supply was initially increased continuously (up to max. 1.4 vvm) and the stirrer speed continuously increased up to a maximum of 1200 rpm.

The fermentation was carried out at a temperature of 32° C. until an optical density of 30 to 40 was attained (measured at 600 nm). This was generally the case after approx. 17 h.

### C. Main Culture (Main Fermenter):

The fermentations were carried out in fermenters of the type BIOSTAT B-DCU from Sartorius Stedim GmbH (Gottingen, D). A culture vessel having a 2 l total volume was used at an initial working volume of 1 l. The fermentation medium comprises the following constituents: 3 g/l (NH₄)₂SO₄, 1.7 g/l KH₂PO₄, 0.25 g/l NaCl, 0.6 g/l MgSO₄×7 H₂O, 0.03 g/l CaCl₂×2 H₂O, 0.15 g/l FeSO₄×7 H₂O, 1 g/l Na citrate×2 H₂O, 15 g/l Cornsteep Dry (CSD) and 3 ml/l trace element solution (consisting of 2.5 g/l H₃BO₃, 0.7 g/l CoCl₂×6 H₂O, 0.25 g/l CuSO₄×5 H₂O, 1.6 g/l MnCl₂×4 H₂O, 0.3 g/l ZnSO₄×7 H₂O, 0.15 g/l Na₂MoO₄×2 H₂O). After sterilization of this basal medium, the following constituents were added under sterile conditions: 10 g/l glucose, 0.018 g/l vitamin B1, 0.09 g/l vitamin B6 and 15 mg/l tetracycline.

100 ml of preculture 2 were transferred to the fermentation vessel to inoculate the main culture. The pH in the fermenter at the start was adjusted to 7.0 by addition of a 25% NH₄OH solution and maintained at this value by automatic correction with 25% NH₄OH. The cultures were stirred at the start at 400 rpm and were flushed with sterilized compressed air via a sterile filter at a gas flow rate of 2 vvm. Under these initial conditions, the oxygen probe had been calibrated to 100% saturation prior to inoculation. The target value for the O₂ saturation during the fermentation was set to 50%. On dropping below the target value for O₂ saturation, a regulation cascade was initiated in order to restore the O₂ saturation to the target value. In this case, the gas supply was initially increased continuously (up to max. 5 vvm) and the stirrer speed continuously increased up to a maximum of 1500 rpm.

The fermentations were carried out at a temperature of 32° C. As soon as the glucose content in the fermenter had decreased from the initial 10 g/l to approx. 2 g/l, 60% glucose solution was added continuously. The feeding rate was adjusted such that the glucose concentration in the fermenter from this point on did not exceed 2 g/l. The glucose determination was carried out using a glucose analyzer from YSI (Yellow Springs, Ohio, USA).

After a fermentation time of 5 h, a sulfur source was supplied in the form of a sterile 1.5 M (NH₄)₂SO₄ stock solution (ammonium sulfate) at a rate of 8-17 mmol/1 per hour (average=11 mmol/l per hour).

After a fermentation time of 16 h, additional glutamate in the form of a sterile 2.3 M potassium glutamate stock solution was added to the main culture at a rate of 7.4 mmol/l per hour.

The fermentation period was 72 hours. Samples were taken at 24 h, 48 h and 72 h and, after reduction with DTT, the proportion of “total γ-glutamylcysteine” in the culture supernatant was determined by HPLC analysis.

TABLE 3
Influence of the gshB deletion and/or gshA overexpression on the
γ-glutamylcysteine production. “Total γ-glutamylcysteine”
(γGC) content after neutralization of the fermentation broth
and subsequent reduction of the oxidation products with dithiothreitol.

| | Total γ-glutamylcysteine (g/l) | | |
|---|---|---|---|
| E. coli strain | 24 h | 48 h | 72 h |
| W3110/pACYC184-LH | 0 | 0 | 0 |
| W3110/pgshAₚ-gshA^{TTG} | 0.2 ± 0.0 | 0.3 ± 0.0 | 0.3 ± 0.0 |
| W3110/ptufBₚ-gshA^{ATG} | 1.2 ± 0.0 | 1.4 ± 0.0 | 1.5 ± 0.1 |
| W3110ΔgshB/pACYC184-LH | 2.2 ± 0.1 | 2.7 ± 0.1 | 2.9 ± 0.2 |
| W3110ΔgshB/pgshAₚ- | 3.4 ± 0.2 | 5.4 ± 0.1 | 5.7 ± 0.3 |
| gshA^{TTG}(*) | | | |
| W3110ΔgshB/ptufBₚ- | 7.2 ± 0.3 | 13.1 ± 0.5 | 14.4 ± 0.3 |
| gshA^{ATG}(*) | | | |

((*)inventive strain)

TABLE 4
Influence of the gshB deletion and gshA overexpression
in combination with an increased L-cystein biosynthesis
capacity on the γ-glutamylcysteine production. “Total
γ-glutamylcysteine” (γGC) content after neutralization
of the fermentation broth and subsequent reduction of the
oxidation products bis-γ-glutamylcystine and γ-glutamylcystine
with dithiothreitol:

| E. coli W3110ΔgshB | Total γ-glutamylcysteine (g/l) | | |
|---|---|---|---|
| with plasmid | 24 h | 48 h | 72 h |
| pgshAₚ-gshA^{TTG}(*) | 3.4 ± 0.2 | 5.4 ± 0.1 | 5.7 ± 0.3 |
| ptufBₚ-gshA^{ATG}(*) | 7.2 ± 0.3 | 13.1 ± 0.5 | 14.4 ± 0.3 |
| ptufBₚ-gshA^{ATG}-serA2040(*) | 8.5 ± 0.5 | 15.1 ± 1.1 | 16.5 ± 1.1 |
| ptufBₚ-gshA^{ATG}-cysE14(*) | 8.6 ± 0.3 | 15.2 ± 1.0 | 17.0 ± 0.9 |
| ptufBₚ-gshA^{ATG}-cysE14- | 10.0 ± 0.7 | 16.6 ± 0.9 | 20.1 ± 1.2 |
| serA2040(*) | | | |
| ptufBₚ-gshA^{ATG}- | 8.7 ± 0.4 | 15.5 ± 0.2 | 16.9 ± 0.6 |
| serA2040-orf306(*) | | | |
| ptufBₚ-gshA^{ATG}-cysE14- | 8.9 ± 0.5 | 15.7 ± 0.8 | 17.5 ± 1.1 |
| orf306(*) | | | |
| ptufBₚ-gshA^{ATG}-cysE14- | 11.6 ± 0.3 | 17.9 ± 0.7 | 21.8 ± 1.0 |
| serA2040-orf306(*) | | | |

((*)inventive strain)

What is claimed is:

1. A prokaryotic microorganism strain capable of overproducing γ-glutamylcysteine, bis-γ-glutamylcystine and γ-glutamylcystine, which can be prepared from a parent strain, wherein said prokaryotic microorganism strain has a reduced cellular glutathione synthetase activity compared to the parent strain, and has a cellular γ-glutamylcysteine synthetase activity which is more greatly increased than in a strain with similarly reduced cellular glutathione synthetase activity.

2. The microorganism strain as claimed in claim 1, wherein the glutathione synthetase activity in the prokaryotic microorganism strain is so reduced that said activity is at most 50% of the glutathione synthetase activity of a corresponding wild-type strain.

3. The microorganism strain as claimed in claim 1, wherein the cellular γ-glutamylcysteine synthetase activity is around at least a factor of 5 higher than in a strain with similarly reduced cellular glutathione synthetase activity.

4. The microorganism strain as claimed in claim 1, wherein the parent strain is a member of the species Escherichia coli or Pantoea ananatis.

5. The microorganism strain as claimed in claim 1, wherein the parent strain is a microorganism strain which has a biosynthetic pathway for γ-glutamylcysteine and an elevated L-cysteine biosynthesis capacity compared to a corresponding parent strain.

6. The microorganism strain as claimed in claim 1, wherein an elevated γ-glutamylcysteine synthetase activity is achieved by increasing a copy number of a gshA gene or a gshA homolog or by using a promoter which leads to an increased expression of gshA.

7. The microorganism strain as claimed in claim 6, wherein a copy number of the gshA gene or the gshA homolog is increased by cloning into plasmid vectors under control of a promoter.

8. The microorganism strain as claimed in claim 6, wherein the gshA gene has a protein sequence in which alanine at position 494 has been exchanged for valine or leucine (A494V or A494L), or serine at position 495 has been exchanged for threonine (S495T).

9. The microorganism strain as claimed in claim 6, wherein a native start codon TTG of the gshA gene has been replaced by ATG.

10. The microorganism strain as claimed in claim 2, wherein the cellular γ-glutamylcysteine synthetase activity is around at least a factor of 5 higher than in a strain with similarly reduced cellular glutathione synthetase activity.

11. The microorganism strain as claimed in claim 10, wherein the parent strain is a member of the species Escherichia coli or Pantoea ananatis.

12. The microorganism strain as claimed in claim 11, wherein the parent strain is a microorganism strain which has a biosynthetic pathway for γ-glutamylcysteine and an elevated L-cysteine biosynthesis capacity compared to a corresponding parent strain.

13. The microorganism strain as claimed in claim 12, wherein an elevated γ-glutamylcysteine synthetase activity is achieved by increasing a copy number of a gshA gene or a gshA homolog or by using a promoter which leads to an increased expression of gshA.

14. The microorganism strain as claimed in claim 13, wherein a copy number of the gshA gene or the gshA homolog is increased by cloning into plasmid vectors under control of a promoter.

15. The microorganism strain as claimed in claim 13, wherein the gshA gene has a protein sequence in which alanine at position 494 has been exchanged for valine or leucine (A494V or A494L), or serine at position 495 has been exchanged for threonine (S495T).

16. The microorganism strain as claimed in claim 13, wherein a native start codon TTG of the gshA gene has been replaced by ATG.

17. A method for overproducing γ-glutamylcysteine (γGC) and derivatives of γGC, γ-glutamylcystine and bis-γ-glutamylcystine, wherein a microorganism strain as claimed in claim 1 is cultured in a fermentation medium, cells are removed from the fermentation medium and a desired products is purified from the fermentation medium.
